(19) United States
(12) Patent Application Publication
(10) Pub. No.: US 2024/0277773 A1
(43) Pub. Date: Aug. 22, 2024

(54) COMPOSITION AND PROCESS FOR AN INFILTRATION AND TRANSPORT MEDIUM FOR ADIPOSE TISSUE CELLS

(71) Applicant: UNIVERSITEIT GENT, Gent (BE)

(72) Inventors: Bernard DEPYPERE, Marke (BE); Phillip BLONDEEL, Merelbeke (BE); Ann-Sophie MADELEYN, Waarschoot/Lievegem (BE); Luc DE BAERDEMAEKER, Lievegem (BE)

(21) Appl. No.: 18/571,533

(22) PCT Filed: Jun. 29, 2022
(86) PCT No.: PCT/EP2022/067869

§ 371 Date: Dec. 18, 2023

(30) Foreign Application Priority Data
Jul. 2, 2021 (EP) 21183435.3

(51) Int. Cl. A61K 35/28; A61K 31/19; A61K 31/191; A61K 33/00; A61K 33/06; C12N 5/0775

(52) U.S. Cl. CPC A61K 35/28; A61K 31/19; A61K 31/191; A61K 33/00; A61K 33/06; C12N 5/0667; C12N 2500/30; C12N 2509/00

(57) ABSTRACT

The present invention relates to the field of medicine, in particular to internal and aesthetic regenerative medicine. More specifically, the present invention relates to a composition suitable for use as a transport medium for adipose tissue and/or cells derived thereof. The invention further relates to a liposuction infiltration formulation and a process to obtain lipoaspirate material.

### FIELD OF THE INVENTION

The present invention relates to the field of medicine, in particular to internal and aesthetic regenerative medicine. More specifically, the present invention relates to a composition suitable for use as a transport medium for adipose tissue and/or cells derived thereof. The invention further relates to a liposuction infiltration formulation and a process to obtain lipoaspirate material.

### BACKGROUND TO THE INVENTION

Adipose tissue-derived stem cells (ASCs) are considered to be the most advantageous for use in tissue engineering and regenerative medicine, including autologous fat grafting for the treatment of soft-tissue deficiencies. Furthermore, these cells have been widely clinically applied in the treatment of a variety of medical conditions, including cardiovascular diseases, auto-immune diseases, neurodegenerative diseases and orthopedic pathology. Due to the abundant subcutaneous localization of white adipose tissue, it is easily accessible without significant donor site morbidity and can be harvested in relatively high quantities using minimally invasive procedures such as liposuction.

The presence of ASCs in the lipoaspirate is crucial for the survival of the transplanted fat graft. Apart from their self-renewal capacity and multipotent differentiation potential, ASCs indirectly enhance angiogenesis and support the differentiation and phenotypic maturation of other cells in their micro-environment. Furthermore, these cells display immunomodulatory, immunosuppressive, and anti-inflammatory properties. For these reasons among others, preserving the viability and survival of these cells is of great importance to secure the therapeutic potential of these cells.

Suction-assisted lipectomy or liposuction is a minimally invasive surgical procedure for the collection of subcutaneous white adipose tissue. Different techniques can be used to perform liposuction, including the dry and tumescent technique. The tumescent technique was initially developed to perform liposuction procedures under local anesthesia, eliminating the need for general anesthesia. This technique involves the infiltration of a ‘wetting’ solution into the subcutaneous adipose tissue until the area to be suctioned becomes swollen and firm (i.e. tumescent). As wetting solution for the tumescent liposuction technique, Klein's tumescent solution (i.e. Klein solution) is most commonly used. This is a dilute anesthetic solution containing lidocaine (local anesthetic), combined with epinephrine (vasoconstrictor to minimize blood loss and absorption of lidocaine), sodium bicarbonate and normal saline (NaCl 0.9%). Unfortunately, autologous fat grafting is associated with unpredictable resorption rates, which often lead to low graft survival and small residual volumes. Firstly, during the liposuction procedure, adipose tissue is completely cut off from the blood supply (i.e. nutrients and oxygen) and after transplantation, the fat graft is not immediately revascularized. Common general practice suggests that acceptable clinical results cannot be obtained with transplant adipose tissue exposed to ischemia in excess of 6 hours. Therefore, rapid and efficient transport and revascularization of the lipoaspirate material is necessary to guarantee graft survival. Secondly, growing evidence suggests that some components of the Klein solution might not be suitable to preserve the lipoasirate material—and associated ASCs—since some components significantly impair cell viability and survival. For example, several studies have reported on the cytotoxic effects of lidocaine on adipocytes, preadipocytes, ASCs and the stromal vascular fraction in the lipoaspirate and the possible implications this may have on clinical outcomes. Moreover, epinephrine contributes to the prolonged duration of lidocaine action. These limitations urge the need for new approaches to improve unpredictable resorption rates and low graft survival.

It was unexpectedly found that a composition of adipose tissue and/or cells derived thereof together with a storage medium comprising sodium acetate significantly prolongs the viability of these cells as well as the survival and differentiation potential. The inventors clearly showed that this storage medium resulted in a higher mean cell count and supported the survival of the lipoaspirate in vitro during the first consecutive 24 to 48 hours which is substantially more than the acceptable ischemic time of (typically) 6 hours. Consequently, this medium supports the lipoaspirate material during transport from the operating room to the laboratory, where the adipose tissue and/or cells derived thereof can even be stored for an additional period of time before further processing.

It is believed that adipose tissue contains one hundred up to one thousand times more multipotent stem cells per cubic centimeter than bone marrow. Therefore, considering the promising therapeutic potential of autologous fat grafting within the field of regenerative medicine and tissue engineering, the present invention also proposes an optimized process and infiltration formulation to obtain lipoaspirate material with high quality ASCs to increase the chance of successful tissue or cell transplantations. More specifically, the proposed formulation comprises components that do not contribute substantially to impaired cell viability and survival as compared to other solutions e.g. Klein solution.

### SUMMARY OF THE INVENTION

In a first aspect, the present invention provides a composition comprising adipose tissue and/or cells derived thereof and sodium acetate (CH₃COONₐ).

In a particular embodiment, the present invention provides a composition comprising CD34+ adipose tissue derived stem cells (CD34+ ASCs) and sodium acetate.

In a specific embodiment of the present invention, said composition further comprises one or more components such as selected from the list comprising sodium, potassium, magnesium, chloride and/or gluconate; and/or salts thereof.

In another specific embodiment, said composition has an osmolality ranging from about 270 to about 310 mOsmol/kg, preferably about 295 mOsmol/kg.

In a specific embodiment, said composition further comprising a thrombin inhibitor.

In another specific embodiment, said thrombin inhibitor is selected from the list comprising: heparin, desirudin, lepirudin, bivalirudin, hirudin, argatroban, inogatran, melagatran, dabigatran.

In yet another specific embodiment, said thrombin inhibitor is heparin, in particular unfractionated heparin and/or low-molecular-weight derivates thereof. In a further embodiment of the present invention, said composition further comprises a protein such as a type of globular protein comprising serum albumin, globulins or a functional equivalent thereof.

In yet a further embodiment, the present invention provides a composition for use in human and/or veterinary medicine, in particular for use in stem-cell based therapies, regenerative medicine, and/or plastic surgery.

In another aspect, the present invention relates to a process for obtaining adipose tissue-derived stem cells (ASCs), comprising the steps of:
a) Obtaining lipoaspirate material;
b) Adding CH₃COONₐ to said lipoaspirate material of step a; thereby obtaining a composition as defined herein;
c) Isolating the stromal vascular fraction (SVF) of said lipoaspirate material of step b;
d) Optionally isolating the ASCs from said SVF of step c.

In another aspect, the present invention relates to a process for obtaining CD34+ adipose tissue-derived stem cells (CD34+ ASCs), comprising the steps of:
a) Obtaining lipoaspirate material;
b) Adding CH₃COONₐ to said lipoaspirate material of step a; thereby obtaining a composition as defined herein;
c) Isolating the stromal vascular fraction (SVF) of said lipoaspirate material of step b;
d) Optionally isolating the CD34+ ASCs from said SVF of step c.

In a specific embodiment, the isolation step d) of said process comprises:
adding said SVF of step c to a bioreactor of which the surface is pre-treated with a culture medium to promote adhesion of the (CD34+) ASCs and subsequently;
detaching said (CD34+) ASCs and collecting the cells in a collection medium.

In a particular embodiment of the process of the present invention, the amount of viable cultured ASC cells is preferably increased by at least between 5% to 40%, when compared to untreated samples or samples treated with conventional solutions such as normal saline.

In yet another aspect, the present invention relates to a formulation comprising CH₃COONₐ, normal saline, and a vasoconstrictor.

In a specific embodiment, the present invention relates to a formulation comprising CH₃COONₐ, normal saline, and a vasoconstrictor for use in liposuction or lipofilling.

In a specific embodiment of the present invention, said formulation further comprises one or more components selected from the list comprising sodium, potassium, magnesium, chloride, and/or gluconate; and/or salts thereof.

In yet another embodiment, said formulation further comprises a protein such as a type of globular protein comprising serum albumin, globulins or functional equivalents thereof.

In a further embodiment, said formulation further comprises a local anesthetic component.

In another aspect, the present invention relates to a combination comprising said formulation and adipose cells, SVF or ASCs.

In yet a further embodiment, the present invention provides a combination for use in human and/or veterinary medicine, in particular for use in stem-cell based therapies, regenerative medicine, and/or plastic surgery.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Isolation procedure of adipose tissue-derived stem cells (ASCs) via mechanical processing of lipoaspirate material.
(1) Adipose tissue is harvested through liposuction (either dry liposuction or liposuction with the proposed wetting solution) and (2) collected in a sterile bag or container together with CH₃COONₐ; thereby obtaining a composition according to the present invention. (3) The stromal vascular fraction (SVF) is mechanically isolated by mixing CH₃COONₐ with the adipose tissue by shifting 30 times through two syringes back-and-forth. (4) After centrifuging the surplus of the CH₃COONₐ comprising solution and cell debris is discharged, leaving only the SVF pellet behind. (5) The SVF pellet is counted using e.g. flow cytometry and divided in doses which can be optionally preserved at −80° C. or (6) upon request of the physician, used directly in e.g. a stem cell-based therapy.

FIG. 2: In vitro experiment to assess cell viability of lipoaspirate with an Alamar Blue assay.
(1) Adipose tissue is harvested through dry liposuction and collected in Luer-lok syringe. (2) The lipoaspirate was centrifuged and the resulting bottom layer, predominantly composed of adipose tissue, was used for the in vitro study. The lipoaspirate material was cultured in 24-well culture plates. (3) A cell culture medium (i.e. composition as defined herein) was added with the lipoaspirate in a ratio of 1:1 and incubated for different time points (t0=within 3 hours of harvesting), t24=24 hours, and t48=48 hours). The plates were incubated at 37° C. for the corresponding periods of time, during which culture media were not changed. (4-6) Cell viability was assessed immediately (t0, i.e. within 3 hours of harvesting), after 24 hours (t24) and 48 hours (t48) using the Alamar Blue assay.

FIG. 3: Cell viability of adipose tissue-derived stem cells (ASCs) derived directly from lipoaspirate material incubated in different solutions. Viability of cells within the lipoaspirate, expressed in mean cell count for different time points. Note that the Y-axis does not start in 0. As compared to NaCl 0.9%, the composition of the invention shows an overall higher cell viability that decreases less rapidly in the first 24 hours. The differences in cell viability between NaCl 0.9% and the composition of the invention was statistically significant after 24 and 48 hours (p<0.05).

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

As further detailed herein below, the present invention is based on the finding that sodium acetate (CH₃COONₐ) significantly increased viability of adipose tissue and/or cells derived thereof. Accordingly, the invention is amongst other directed to the following aspects:
Compositions comprising (CD34+) adipose tissue and/or cells derived thereof and sodium acetate
Formulations comprising sodium acetate, normal saline and a vasoconstrictor

As used herein and unless otherwise specified, the terms “composition” or “formulation” refer to the identity of all different, individual substances, compounds or elements (e.g. agents, modulators, regulators, etc.) that constitute the composition or formulation. It can be a solution, mixture, an emulsion, a suspension, liquid, aqueous or non-aqueous formulations or any combination thereof prepared according to a specific procedure. Some components impart specific properties to the composition or formulation when it is put into use. The compositions or formulations are preferably pharmaceutical components, comprising one or more pharmaceutically excipients, carriers, diluents.

In particular, as defined herein, the term ‘composition’ is in particular used in the context of the combination of adipose tissue and/or cells derived thereof and sodium acetate. This composition can for example be obtained by adding sodium acetate to a lipoaspirate material; and is in particular intended for prolonging viability of the adipose tissue and/or derived cells contained in said composition.

On the other hand, the term ‘formulation’ is used in the context of a combination of sodium acetate, normal saline and a vasoconstrictor. This formulation is particularly suitable as a wetting solution for obtaining a lipoaspirate, which then already contains such sodium acetate upon retrieval from a patient. The thus obtained lipoaspirate, i.e. containing the formulation (sodium acetate, normal saline and vasoconstrictor) as well as adipose tissue and/or cells derived therefrom is herein referred to as ‘combination’.

Accordingly, in a first aspect, the present invention thus in particular relates to (i) a composition suitable for use as a transport medium for (CD34+) adipose tissue and/or cells derived thereof. The inventors of the present invention have found that a composition of adipose tissue and/or cells derived thereof together with sodium acetate significantly prolongs the viability of these cells as well as the survival and differentiation potential. The major advantage of this invention is that this medium supports the survival of the lipoaspirate in vitro during the first consecutive 24 to 48 hours which is substantially longer than the acceptable ischemic time of (typically) 6 hours. This innovative solution prolongs the time window to transport the lipoaspirate material from the operating room to the laboratory, where the adipose tissue can be stored or further processed.

A second and third aspect of the present invention relates to (ii) an infiltration formulation and (iii) an optimized process to obtain lipoaspirate material with high quality ASCs to increase the chance of successful tissue or cell transplantations. The inventors of the present invention have found that using the proposed formulation increases overall viable cell count, limits cytotoxic effects of e.g. lidocaine and improves resorption rates and low graft survival.

Accordingly and as already detailed herein above, in a first aspect, the present invention provides a composition comprising adipose tissue and/or cells derived thereof and sodium acetate (CH₃COONₐ).

In the context of the present invention, the term “sodium acetate” or “CH₃COONₐ” or “NaOAc” relates to a salt of weak acid (i.e. acetic acid, CH₃COOH) and strong base (i.e. sodium hydroxide, NaOH). When dissolved in water, it dissociates into sodium (Na+) cations and acetate (CH₃COO) anions. Subsequently, the acetate anion undergoes hydrolysis and captures one hydrogen (H+) ion from water, forming acetic acid. As used herein, a solution of sodium acetate (a basic salt of acetic acid) and acetic acid can act as a buffer to keep a relatively constant pH level in a mildly acidic range.

In some embodiments, said composition, formulation or combination as defined herein may comprise acetate (CH₃COO⁻) with a minimum amount of at least about 20, 21, 22, 23, 24, 25, 26, 27, 28, 29, 30, 31, 32, 33, 34, 35, 36, 37, 38, 39, 40 mmol/liter. In a particular embodiment said composition, formulation or combination may comprise acetate with an amount of about 27 mmol/liter.

In another embodiment, the viability of said adipose tissue and/or cells derived thereof, in particular adipose tissue-derived stem cells (ASCs) in the composition, or combination is maintained for at least about 2 hours, such as at least about 3, 4, 5, 6, 7, 8, 9, 10, 11 hours, in particular at least about 12 hours such as at least about 13, 14, 15, 16, 17, 18, 19, 20, 21, 22, 23 hours, in particular at least about 24 hours, such as at least about 25, 26, 27, 28, 29, 30, 31, 32, 33, 34, 35 hours, in particular at least about 36 hours, such as at least about 37, 38, 39, 40, 41, 42, 43, 44, 45, 46, 47, 48 hours.

In yet another embodiment, the viability of said adipose tissue and/or cells derived thereof, in particular adipose tissue-derived stem cells (ASCs) in the composition or combination as defined herein is maintained for a least about 2 days, such as at least about 3 days.

As used herein, the term “viability” is referred to as a measure of the proportion of live, healthy cells within a population and is usually expressed as mean cell count for a given time period. Cell viability assays are used to determine the overall health of cells, optimize culture or experimental conditions, and to measure cell survival following treatment with compounds. Typically, cell viability assays such as alamarBlue assay provide a readout of cell health through measurement of metabolic activity, ATP content, or cell proliferation.

In a specific embodiment, the amount of viable cells is preferably increased by at least 5%, such as at least 10%, at least 15%, at least 20%, at least 25%, at least 30%, at least 35%, at least 40%, when compared to untreated samples or samples treated with conventional solutions such as normal saline as used in the example part.

In some embodiments, the amount of viable cells after at least 3 hours of incubation is preferably increased by at least 5%, such as at least 10%, at least 15%, at least 20%, at least 25%, at least 30%, at least 35%, at least 40%, when compared to untreated samples or samples treated with conventional solutions such as normal saline as used in the example part.

In some embodiments, the amount of viable cells after at least 6 hours of incubation is preferably increased by at least 5%, such as at least 10%, at least 15%, at least 20%, at least 25%, at least 30%, at least 35%, at least 40%, when compared to untreated samples or samples treated with conventional solutions such as normal saline as used in the example part.

In some embodiments, the amount of viable cells after at least 12 hours of incubation is preferably increased by at least 5%, such as at least 10%, at least 15%, at least 20%, at least 25%, at least 30%, at least 35%, at least 40%, when compared to untreated samples or samples treated with conventional solutions such as normal saline as used in the example part.

In some embodiments, the amount of viable cells after at least 24 hours of incubation is preferably increased by at least 5%, such as at least 10%, at least 15%, at least 20%, at least 25%, at least 30%, at least 35%, at least 40%, when compared to untreated samples or samples treated with conventional solutions such as normal saline as used in the example part.

In some embodiments, the amount of viable cells after at least 36 hours of incubation is preferably increased by at least 5%, such as at least 10%, at least 15%, at least 20%, at least 25%, at least 30%, at least 35%, at least 40%, when compared to untreated samples or samples treated with conventional solutions such as normal saline as used in the example part.

In some embodiments, the amount of viable cells after at least 48 hours of incubation is preferably increased by at least 5%, such as at least 10%, at least 15%, at least 20%, at least 25%, at least 30%, at least 35%, at least 40%, when compared to untreated samples or samples treated with conventional solutions such as normal saline as used in the example part. In particular, the present invention provides a composition or combination wherein said adipose tissue cells comprise adipose cells and stromal vascular fraction (SVF), further comprising adipose tissue-derived stem cells (ASCs), hematopoietic and endothelial progenitor cells, immune cells, fibroblasts, pericytes and other stromal components.

As used herein, the term “adipose tissue” is a loose connective tissue which mainly consists of mature adipocytes, stromal vascular fraction cells, blood vessels, lymph nodes and nerves. The stromal vascular fraction (SVF) contains mesenchymal stem cells (i.e. adipose tissue-derived stem cells), preadipocytes, hematopoietic and endothelial progenitor cells, immune cells (i.e. macrophages and monocytes), pericytes, and fibroblasts. Four types of mature adipocytes can be distinguished: white adipocytes, brown adipocytes and the more recently discovered beige and perivascular adipocytes.

Adipose tissue-derived stem cells are considered to be ideal for application in regenerative therapies. Their main advantage over mesenchymal stem cells derived from other sources, e.g. from bone marrow, is that they can be easily and repeatable harvested using minimally invasive techniques with low morbidity. ASCs are multipotent and can differentiate into various cell types of the tri-germ lineages, including e.g. osteocytes, adipocytes, neural cells, vascular endothelial cells, cardiomyocytes, pancreatic B-cells, and hepatocytes.

In a particular embodiment, the present invention provides a composition comprising CD34+ adipose tissue derived stem cells (CD34+ ASCs) and sodium acetate.

Unless otherwise specified, the invention is directed to a composition comprising ASCs and sodium acetate, wherein the ASCs can be CD34+ cells. Accordingly, when reference is made to ASCs, it may also refer to CD34+ ASCs.

As used herein, the term “CD34” is to be understood as a transmembrane phosphoglycoprotein protein which is commonly used as a marker for identifying human haemopoietic stem cells. Cells expressing CD34 (CD34+ cell) are often used clinically to quantify the number of haemopoietic stem cells for use in haemopoietic stem cell transplantation. In the adipose tissue, adipose mesenchymal stem/stromal cells are also characterized by the expression of CD34. Its expression is gradually lost upon standard ASC expansion in vitro. The CD34+ cell count is used to monitor the quality of harvested cells and to provide an indication as to the likelihood of the collection being sufficient for engraftment. For SVF mechanical isolation, a dose of 10\hat{ }4 CD34+ cells/ml fat is preferred for successful engraftment.

The inventors have observed beneficial effects of a thrombin inhibitor, in particular heparin, on the mean cell count of CD34+ ASCs and thus clinical manifestation of fat engraftment. The enrichment in CD34+ ASCs as shown in the examples, demonstrate that a composition further comprising heparin increases harvested stem cell numbers significantly prolonging viability and survival of these cells.

In a specific embodiment, said composition further comprising a thrombin inhibitor.

As used herein, the term “thrombin inhibitor” is to be understood as a class of medication that act by either indirectly or directly inhibiting the enzyme thrombin (factor 11a). Thrombin inhibitors inactivate free thrombin and also the thrombin that is bound to fibrin. Within the context of the present invention, the thrombin inhibitor can be an indirect thrombin inhibitor such as for example heparin or it can be a direct thrombin inhibitor such as for example desirudin, lepirudin, bivalirudin, hirudin.

In another specific embodiment, said thrombin inhibitor is selected from the list comprising: heparin, desirudin, lepirudin, bivalirudin, hirudin, argatroban, inogatran, melagatran, dabigatran.

In another specific embodiment, the thrombin inhibitor is heparin, in particular unfractionated heparin and/or low-molecular-weight derivates thereof.

In some clinical applications, (unfractionated) heparin and its low-molecular-weight derivatives (e.g., enoxaparin, dalteparin, tinzaparin) are effective in preventing deep vein thromboses and pulmonary emboli in people at risk. Unfractionated heparin is commonly used in hemodialysis. Comparing to low-molecular-weight heparin, unfractionated heparin does not have prolong anticoagulation action after dialysis, and low cost.

In accordance with an embodiment of the present invention, said composition or combination comprises a concentration of heparin ranging from about 0.01 to about 70% w/v, in particular from about 1 to about 50% w/v, in particular about 5 to about 25%, more particular about 10%.

In some embodiments, said composition, or combination may comprise heparin with a minimum of at least about 0.01, 0.05, 0.10, 0.15, 0.20, 0.25, 0.30, 0.35, 0.40, 0.45, 0.50, 0.55, 0.60, 0.65, 0.70, 0.80, 0.85, 0.90, 0.95% (w/v), in particular at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% (w/v).

In case a combination of unfractionated heparin and its low-molecular-weight derivatives are used, the ratio unfractionated heparin and low-molecular-weight derivative may range from about 1:100, 1:50, 1:25 to about 100:1, 50:1, 25:1, preferably from about 1:10, 1:5, 1:2 to about 10:1, 5:1, 2:1 and more preferably 1:1. In a specific embodiment of the present invention, said composition, formulation or combination further comprises one or more components such as selected from the list comprising sodium, potassium, magnesium, chloride, and/or gluconate; and/or salts thereof. In a particular embodiment, said components are selected from the list comprising sodium chloride, potassium chloride, magnesium chloride hexahydrate, and/or sodium gluconate.

In a particular embodiment, said composition, formulation or combination comprise sodium acetate in combination with sodium chloride; alternatively sodium acetate in combination with potassium chloride; alternatively sodium acetate in combination with magnesium chloride hexahydrate; alternatively sodium acetate in combination with sodium gluconate; alternatively sodium acetate in combination with sodium chloride, potassium chloride; alternatively sodium acetate in combination with sodium chloride, magnesium chloride hexahydrate; alternatively sodium acetate in combination with sodium chloride, sodium gluconate; alternatively sodium acetate in combination with potassium chloride, magnesium chloride hexahydrate; alternatively sodium acetate in combination with potassium chloride, sodium gluconate; alternatively sodium acetate in combination with magnesium chloride hexahydrate, sodium gluconate; alternatively sodium acetate in combination with sodium chloride, potassium chloride, magnesium chloride hexahydrate; alternatively sodium acetate in combination with sodium chloride, potassium chloride, sodium gluconate; alternatively sodium acetate in combination with sodium chloride, magnesium chloride hexahydrate, sodium gluconate; alternatively sodium acetate in combination with potassium chloride, magnesium chloride hexahydrate, sodium gluconate; alternatively sodium acetate in combination with sodium chloride, potassium chloride, magnesium chloride hexahydrate, sodium gluconate.

In some embodiments, said composition, formulation or combination may comprise sodium with a minimum amount of at least about 100, 110, 120, 130, 140, 150, 160, 170, 180 mmol/liter. In a particular embodiment said composition or formulation may comprise sodium with an amount of about 140 mmol/liter.

In some embodiments, said composition, formulation or combination may comprise potassium with a minimum amount of at least about 1, 2, 3, 4, 5, 6, 7, 8, 9 mmol/liter. In a particular embodiment said composition or formulation may comprise potassium with an amount of about 5 mmol/liter.

In some embodiments, said composition, formulation or combination may comprise magnesium with a minimum amount of at least about 1.1, 1.2, 1.3, 1.4, 1.5, 1.6, 1.7, 1.8, 1.9, 2.0 mmol/liter. In a particular embodiment said composition or formulation may comprise magnesium with an amount of about 1.5 mmol/liter.

In some embodiments, said composition, formulation or combination may comprise chloride with a minimum amount of at least about 90, 92, 94, 96, 98, 100, 102, 104, 106, 108, 110, 120, 125, 130, 135, 140, 145, 150, 155, 160 mmol/liter. In a particular embodiment said composition or formulation may comprise chloride with an amount of about 98 mmol/liter.

In some embodiments, said composition, formulation or combination may comprise gluconate (C₆H₁₁O₇) with a minimum amount of at least about 15, 16, 17, 18, 19, 20, 21, 22, 23, 24, 25, 26, 27, 28, 29, 30 mmol/liter. In a particular embodiment said composition or formulation may comprise gluconate with an amount of about 23 mmol/liter.

In a specific embodiment, the composition, formulation or combination comprises preferably about 27 mmol/liter acetate, about 140 mmol/L sodium, about 5 mmol/L potassium, about 1.5 mmol/L magnesium, about 98 mmol/L chloride, and about 23 mmol/L of gluconate.

In another specific embodiment, said composition, formulation or combination has an osmolality ranging from about 270 to about 310 mOsmol/kg, preferably about 295 mOsmol/kg.

In yet another embodiment, said composition or combination of the present invention may comprise lipoaspirate and CH₃COONₐ or a CH₃COONₐ solution in a ratio of lipoaspirate:CH₃COONₐ from about 1:100, 1:50, 1:25 to about 100:1, 50:1, 25:1, preferably from about 1:10, 1:5, 1:2 to about 10:1, 5:1, 2:1 and more preferably 1:1.

In yet another embodiment, said combination may comprise lipoaspirate and a formulation as defined herein in a ratio lipoaspirate:formulation from about 1:100, 1:50, 1:25 to about 100:1, 50:1, 25:1, preferably from about 1:10, 1:5, 1:2 to about 10:1, 5:1, 2:1 and more preferably 1:1.

According to the present invention, the term “osmolality” refers to the measure of a solute concentration or the number of osmoles of solute particles per unit volume of solution (osmol/kg). Osmolality is—in contrast to osmolarity (osmol/L)—not affected by changes in water content, temperature and pressure. The osmotic pressure of a solution determines how a solvent will diffuse across a semipermeable membrane (osmosis) that separates two solutions of different osmotic concentrations.

In some embodiments, said composition, formulation or combination has an osmolality ranging from about 260 to about 315 mOsmol/kg, in particular from about 270 to about 305 mOsmol/kg, more in particular from about 280 to about 295 mOsmol/kg.

In yet a further embodiment of the present invention, said composition, formulation or combination has a pH ranging from about 6.5 to about 8.0, preferably about pH 7.4.

In some embodiments, said composition, formulation or combination has a pH of at least about 6.5, 6.6, 6.7, 6.8, 6.9, 7.0, 7.1, 7.2, 7.3, 7.4, 7.5, 7.6, 7.7, 7.8, 7.9, 8.0, preferably a pH of about 7.4.

In a specific embodiment, said composition, formulation or combination has a strong ion difference (SID) of about 20 to about 50 mEq/L, about 30 to about 50 mEq/L, about 40 to about 50 mEq/L, preferably about 50 mEq/L.

As used herein, the term “strong ion difference (SID)” is to be understood as the difference between the positively and negatively charged strong ions in plasma. Strong cations predominate in the plasma at physiologic pH leading to a net positive plasma charge of approximately +40-45. A SID of 50 is considered an “alkalinizing” solution leading to an increase plasma pH after administration.

In a very specific embodiment of the present invention, said composition comprises:
adipose tissue and/or cells derived therefrom
at least about 20 mmol/L acetate,
about 140 mmol/L sodium,
about 5 mmol/L potassium,
about 1.5 mmol/L magnesium,
about 98 mmol/L chloride,
about 23 mmol/L gluconate.

In a very specific embodiment of the present invention, said formulation comprises:
at least about 20 mmol/L acetate,
about 9 g/L saline,
about 1 mg/L vasoconstrictor.

In another very specific embodiment of the present invention, said combination comprises:
adipose tissue and/or cells derived therefrom,
at least about 20 mmol/L acetate,
about 9 g/L saline,
about 1 mg/L vasoconstrictor.

In a further embodiment of the present invention, said composition, formulation or combination may further comprise a protein such as a type of globular protein comprising serum albumin, globulins, or functional equivalents thereof as well as non-globular proteins comprising fibrinogen, platelets. In particular, said composition, formulation or combination may comprise other components such as water, salts, lipids, hormones, antibiotics and/or gelatins.

As used herein, the term “globular proteins” relate to hemoglobin, the alpha, beta and gamma (IgA, IgD, IgE, IgG and IgM) globulin. More specifically, nearly all enzymes with major metabolic functions are globular in shape, as well as many signal transduction proteins. Albumins are also globular proteins, although, unlike other globular proteins, they are completely soluble in water. Human serum albumin (HAS) is a sterile, non-pyrogenic preparation of serum albumin that can be obtained by fractionating blood, plasma or serum from healthy human donors. Albumin buffers pH among other functions. HSA has been found advantageous to neutralize anesthetics such as lidocaine, tetracaine, bupivacaine. In particular, lidocaine and HAS co-crystallize as a dimer in an unusual space group. The dimer consists of one HSA molecule without ligand and one HSA molecule with a single, bound lidocaine. Furthermore, HAS is often used to replace lost fluid and help restore blood volume in trauma, burns and surgery patients.

In the context of the present invention, structurally equivalents of human serum albumin comprise recombinant human albumin (rHA), bovine serum albumin, fetal bovine serum. Recombinant human albumin is a highly purified animal-, virus-, and prion-free product. In a particular embodiment, said formulation used for human medicine applications preferably does not contain non-human derived albumins such as bovine serum albumin or fetal bovine serum.

In this case, the formulation is preferably xeno-free, meaning that it can comprise human-derived components, but no components from animals other than humans. Accordingly, said composition, formulation or combination may comprise non-human derived albumins if it is used for non-human medicine applications.

In accordance with an embodiment of the present invention, said composition, formulation or combination comprises a concentration of serum albumin or equivalent thereof ranging from about 0.01 to about 70% w/v, in particular from about 1 to about 50% w/v, in particular about 5 to about 25%, more particular about 10%.

In some embodiments, said composition, formulation or combination may comprise human serum albumin with a minimum of at least about 0.01, 0.05, 0.10, 0.15, 0.20, 0.25, 0.30, 0.35, 0.40, 0.45, 0.50, 0.55, 0.60, 0.65, 0.70, 0.80, 0.85, 0.90, 0.95% (w/v), in particular at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% (w/v).

In some embodiments, said composition, formulation, or combination may comprise recombinant human albumin with a minimum of at least about 0.01, 0.05, 0.10, 0.15, 0.20, 0.25, 0.30, 0.35, 0.40, 0.45, 0.50, 0.55, 0.60, 0.65, 0.70, 0.80, 0.85, 0.90, 0.95% (w/v), in particular at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% (w/v).

In some embodiments, said composition, formulation or combination may comprise bovine serum albumin with a minimum of at least about 0.01, 0.05, 0.10, 0.15, 0.20, 0.25, 0.30, 0.35, 0.40, 0.45, 0.50, 0.55, 0.60, 0.65, 0.70, 0.80, 0.85, 0.90, 0.95% (w/v), in particular at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% (w/v).

In some embodiments, said composition, formulation or combination may comprise fetal bovine serum with a minimum of at least about 0.01, 0.05, 0.10, 0.15, 0.20, 0.25, 0.30, 0.35, 0.40, 0.45, 0.50, 0.55, 0.60, 0.65, 0.70, 0.80, 0.85, 0.90, 0.95% (w/v), in particular at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% (w/v).

In another aspect, the present invention relates to a process for obtaining adipose tissue-derived stem cells (ASCs), comprising the steps of:
a) Obtaining lipoaspirate material;
b) Adding CH₃COONₐ to said lipoaspirate material of step a; thereby obtaining a composition as defined herein;
c) Isolating the stromal vascular fraction (SVF) of said lipoaspirate material of step b;
d) Optionally isolating the ASCs from said SVF of step c.

Moreover, the process described herein is also applicable for obtaining CD34+ adipose tissue-derived stem cells (ASCs). Thus, when reference is made to adipose tissue-derived stem cells (ASCs) it can also refer to CD34+ ASCs and vice versa.

For the avoidance of doubt, the components, concentrations, ratio's, . . . defined herein above for the compositions, formulations and combinations of the present invention, equally applies to the processes defined herein.

In the context of the process of the present invention, adipose tissue-derived stem cells (ASCs) or CD34+ ASCs are obtained (and further processed) according to a procedure summarized in FIG. 1. In brief, (1) the lipoaspirate material harvested during liposuction (2) is collected in a sterile bag or container together with CH₃COONₐ (or alternatively the CH₃COONₐ is added to the bag or container containing the lipoaspirate); thereby obtaining a composition according to the present invention. (3) The stromal vascular fraction (SVF) is mechanically isolated by mixing CH₃COONₐ with the adipose tissue and shifting the fluids through two syringes back-and-forth. (4) After centrifuging the surplus of the CH₃COONₐ comprising solution, cell debris is discharged, leaving only the SVF pellet behind. (5) The SVF pellet is counted using e.g. flow cytometry and divided in doses which can be optionally preserved at −80° C. or (6) upon request of the physician, used directly in e.g. a stem cell-based therapy.

As used herein, the term “lipoaspirate material” is to be understood as material removed by lipoaspiration and comprises human adipose tissue which mainly consists of mature adipocytes, stromal vascular fraction cells, blood vessels, lymph nodes and nerves.

In the context of the process of the present invention, lipoaspirate material can be harvested in a container from the abdomen, flanks, thighs, hips, medial knees, upper arms, chest, buttocks and back by any kind of minimally invasive surgical liposuction procedure. In the context of the present invention, the term “liposuction” comprises dry liposuction, wet liposuction, superwet liposuction, tumescent liposuction.

As used herein, the term “dry liposuction” relates to a procedure that requires general anesthesia wherein the aspiration cannula is inserted directly into the space from which adipose tissue is to be removed, without prior infiltration of a wetting solution causing excessive intraoperative bleeding and tissue injury (blood loss 20-45% of the aspirated volume).

As used herein, the term “wet liposuction” relates to a procedure that also requires general anesthesia in addition to local anesthesia, wherein adipose tissue is injected with a wetting solution, which helps to dissolve tissue holding the fat, prior to suctioning. Wet liposuction technique causes less blood loss (approximately 3% to 30% of the aspirated volume) compared to the dry technique

As used herein, the term “superwet liposuction” relates to a procedure that requires general anesthesia in addition to local anesthesia, wherein adipose tissue is injected with a wetting solution in a ratio of 0.5 to 1.5 ml per milliliter of injectate to lipoaspirate. Blood loss is approximately 1 to 8% of the aspirated volume.

As used herein, the term ‘tumescent liposuction’ relates to a technique involving the infiltration of large volumes of wetting solution (ratio 3-4 mL per milliliter of injectate to lipoaspirate) with dilute local anesthetic into the subcutaneous adipose tissue until the area to be suctioned becomes swollen and firm (i.e. tumescent). The increased interstitial pressure further spreads the wetting solution through the adjacent tissues, facilitating liposuction. After infiltration of the wetting solution, negative pressure liposuction is performed using blunt-tip cannula's which are inserted through stab incisions. Blood loss is approximately 1% of the aspirated volume.

As used herein, the term “wetting solution” is to be understood as any solution that is able to dissolve tissue holding the fat, prior to suctioning. The infiltration of a ‘wetting’ solution into the subcutaneous adipose tissue results in a swollen and firm area to be suctioned (i.e. tumescent). The most common used wetting solutions are Klein's solution and Hunstad's solution.

In a particular embodiment of the present invention, said formulation (e.g. used as a wetting solution) may further comprise one or more of hyaluronidase, a local anesthetic such as lidocaine, sodium lactate, sodium bicarbonate, or any combination thereof.

As used herein and unless otherwise specified, the term “container” is to be understood as any receptacle or enclosure for holding a product that keeps the product protected by being inside of its structure and is preferably sterile. The container can be made up of glass, plastic, or any other material and is used for storage, package, transport of human-derived material and may be in the form of for example a bag, jar, vial, bottle, vessel, canister, flask, preferably a sterile bag.

In a specific embodiment, the lipoaspirate material is collected in a container—and may optionally be transferred to a smaller container such as a falcon tube—and subsequently CH₃COONₐ or a CH₃COONₐ-containing solution is added to said container filled with lipoaspirate material to obtain said composition. Alternatively, the lipoaspirate is added to a container containing the CH₃COONₐ or a CH₃COONₐ-containing solution to obtain said composition.

Moreover, in another embodiment, the lipoaspirate material is collected using said formulation and transferred to a container thereby obtaining a combination as defined herein, of which the stromal vascular fraction (SVF) may be isolated and further processed.

In a particular embodiment, said composition or combination may be incubated at a temperature between about 10° C. and about 50° C., preferably about 20° C. and about 40° C., most preferably about 37° C.

In another particular embodiment, said composition or combination may be incubated for at least about 1 minute, preferably such as at least about 30 minutes, more preferable at least about 1 hour, most preferable at least about 3 hours, at least about 6 hours, at least about 12 hours, at least about 18 hours, at least about 24 hours, at least about 48 hours, at least about 72 hours.

In some embodiments of the present invention, the stromal vascular fraction (SVF) is isolated from said composition or combination. In particular, as used in the examples part, said composition or combination may be mixed by shuffling said composition or combination back-and-forth between the two Luer-Lock syringes (Luer-to-Luer) for about and between 1 to 50 times, preferably about and between 10 to 40 times, more preferably about and between 20 to 30 times, most preferably about 30 times.

In some embodiments of the present invention, the SVF is isolated from said composition or combination by centrifuging the container at about and between 1000 and 5000 rpm, preferably at about and between 1500 and 4000 rpm, more preferably at about and between 2000 and 3700 rpm, in particular at about 3000 rpm as exemplified in the examples.

In another embodiment of the present invention, the SVF is isolated from said composition or combination by centrifuging the container for about and between 1 and 60 minutes, preferably for about and between 2 and 30 minutes, more preferably for about and between 3 and 10 minutes, in particular for about 7 minutes as exemplified in the examples.

As used herein and unless otherwise specified, the term “centrifugation” is to be understood as rotating said container around its axis to form different layers comprising an oil phase, adipose phase, an infranatant phase (blood, water, aqueous solution) and the SVF pellet.

In a specific embodiment of the present invention, the SVF is isolated from said composition or combination by an enzymatic technique such as collagenase digestion.

In a specific embodiment, said SVF may be cryopreserved for multiple use with a cryoprotectant at a temperature of at least about −40° C., preferably at least about −60° C., most preferably at least about −80° C. As used herein and unless otherwise specified, the term “cryoprotectant” is to be understood as a penetrating or non-penetrating substance used to protect a composition and its associated compounds from freezing damage (i.e. that due to ice formation). Conventional cryoprotectants are commonly glycols (alcohols containing at least two hydroxyl groups) and sugars, such as ethylene glycol, propylene glycol, glycerol and trehalose. In particular, DMSO is added to cell media to reduce ice formation and thereby prevent cell death during the freezing process.

In some embodiment, said SVF may be cryopreserved with DMSO with a minimum amount of at least about 1, 2, 3, 4, 5, 6, 7, 8, 9, 10, 11, 12, 13, 14, 15, preferably about 10% DMSO.

In a specific embodiment of the present invention, the isolation of ASCs from said SVF as used in the examples part comprises:
adding said SVF of step c to a bioreactor of which the surface is pre-treated with a culture medium to promote adhesion of the ASCs and subsequently;
detaching said ASCs and collecting the cells in a collection medium.

In the context of the present invention, the “culture medium” is to be understood as a cell culture medium suitable for maintaining cell viability comprising at least one component or substrate that supplies essential nutrients (amino acids, carbohydrates, vitamins, minerals), a balanced salt solution, pH buffers, hormones, Penicillin/Streptomycin (P/S) and growth factors such as human platelet lysate (hPL), fetal bovine serum (FBS), bovine calf serum, equine serum, and porcine serum. As exemplified in the examples part, said SVF pellet may be resuspended in culture medium.

As used herein, the term ‘bioreactor’ refers to any manufactured device or system designed to grow cells or tissues in the context of cell culture. Cells growing in bioreactors may be submerged in liquid culture medium or may be attached to the surface of a solid culture medium.

In a certain embodiment of the present invention, ASCs can be cultured for at least about 30 minutes, such as at least about 1 day, at least about 1 week, at least about 2 weeks, at least about 3 weeks, at least about 4 weeks, at least about 5 weeks, at least about 6 weeks, at least about 7 weeks, at least about 8 weeks.

In some embodiments, said ASCs may be cryopreserved for multiple use with a cryoprotectant at a temperature of at least about −40° C., preferably about −60° C., most preferably about −80° C.

In the context of the present invention, collected ASCs may be used for other applications comprising for use in human and/or veterinary medicine; in particular for use in stem-cell based therapies, regenerative medicine, and/or plastic surgery.

In yet another aspect, the present invention relates to a formulation comprising sodium acetate (CH₃COONₐ), normal saline, and a vasoconstrictor.

In a specific embodiment, the present invention relates to a formulation comprising CH₃COONₐ, normal saline, and a vasoconstrictor for use in liposuction or lipofilling.

As used herein, the term “normal saline” also refers to “NaCl 0.9%” or “physiological salt” or “physiological saline” or “isotonic saline” and is meant to be a mixture of sodium chloride in sterile water with about 0.90% w/v of NaCl, 308 mOsm/L or 9.0 g per liter. When dissolved in water, sodium chloride dissociates into equal amounts of sodium (Na+) and chloride (CI) ions (i.e. 154 mmol/L each). Normal saline is used as excipient to dissolve other compounds of said formulation such as CH₃COONₐ.

In some embodiments, said formulation may comprise other forms of saline such as half-normal saline (0.45%), quarter-normal saline (0.22%), hypertonic saline (e.g. 3%, 5%, 7%), or any other concentration. In a further embodiment, said formulation may comprise saline with a minimum amount of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0%.

As used herein and unless otherwise specified, the term “vasoconstrictor” is to be understood as a medicine to increase blood pressure or to reduce local blood flow. Vasoconstrictors mixed with local anesthetics are used to increase the duration of local anesthesia by constricting the blood vessels, thereby safely concentrating the anesthetic agent for an extended duration, as well as reducing hemorrhage.

In a certain embodiment, said formulation may comprise a vasoconstrictor with a minimum of at least about 0.1, 0.2, 0.3, 0.4, 0.5, 0.6, 0.7, 0.8, 0.9, 1, 2, 3, 4, 5, 6, 7, 8, 9, 10 μg/ml. As described in the example, said formulation may be prepared by adding 1 mg of epinephrine (1 ml of 1:1000 epinephrine) and 27 mmol/liter acetate to 1000 ml of normal saline.

In another embodiment, said vasoconstrictor of the formulation comprises epinephrine or phenylephrine.

In yet another embodiment, said formulation further comprises a local anesthetic such as lidocaine.

As used herein, the term “local anesthetic” is to be understood as a medication that causes absence of pain in a specific location of the body without a loss of consciousness, as opposed to a general anesthetic. Local anesthetics can be divided into esters (e.g. procaine and tetracaine), which are generally shorter acting and more toxic, and amides (e.g. lidocaine, bupivacaine, ropivacaine and mepivacaine) based on the bridge separating the hydrophobic aromatic ring and the hydrophilic tertiary amine group.

As used herein, the term “lidocaine” also referred to as “lignocaine” relates to a local anesthetic of the amino amide type. Lidocaine mixtures may also be applied directly to the skin or mucous membranes to numb the area and is often used mixed with a small amount of adrenaline (epinephrine) to prolong its local effects and to decrease bleeding. Lidocaine is an integral part of most wetting solutions used in liposuction (e.g. Klein's solution). Lidocaine is used in the wetting solution even when the procedure is performed under epidural or general anesthesia. Since lidocaine metabolism is predictable and the toxicity profile is among the most favorable, it is frequently used in tumescent solutions for liposuction, especially when combined with epinephrine. While most local anesthetics are weak bases, lidocaine is acidified with hydrochloric acid to increase the solubility for storage. Therefore, the pH of commercial lidocaine solutions is 6.5 for lidocaine alone and 4.15 for lidocaine premixed with epinephrine.

In a specific embodiment, said local anesthetic component may comprise lidocaine with a minimum of at least about and between 0.001% to 0.01%, about and between 0.01% and 0.1%, about and between 0.1% and 1%, about and between 1% and 2%, preferably about 1%.

In another aspect, the present invention relates to a combination comprising said formulation and adipose cells, SVF or ASCs.

In yet a further embodiment, the present invention provides a composition, formulation or combination for use in human and/or veterinary medicine.

In particular, the present invention provides a composition or combination for use in stem-cell based therapies.

As used herein, the term “stem cell-based therapies” relate to any treatment for a disease or a medical condition that fundamentally involves the use of any type of viable human stem cells including, embryonic stem cells (ESCs), induced pluripotent stem cells and adult stem cells such as adipose tissue-derived stem cells for autologous (self-derived) and allogeneic (nonself, donor-derived) therapies.

In the context of the present application, the terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptoms but has not yet been diagnosed as having it; (b) inhibiting the disease symptoms, i.e. arresting its development; or (c) relieving the disease symptom, i.e. causing regression of the disease or symptom.

In a further embodiment, said composition or combination can be used in stem-cell based therapies relating to the treatment of a neurodegenerative disease selected from the list comprising: Parkinson's disease, Alzheimer's disease, Huntington's disease, multiple sclerosis, myotrophic lateral sclerosis, multiple system atrophy, (traumatic) spinal cord injury, stroke, hearing loss, (traumatic) brain injury, erectile dysfunction, blindness.

In a further embodiment, the present invention relates to a composition or combination for use in regenerative medicine, and/or plastic surgery.

As used herein, the term “regenerative medicine” relate to the process of replacing, engineering or regenerating human or animal cells, tissues or organs to restore or establish normal function. More specifically, it relates to engineering tissues and organs by stimulating the body's own repair mechanisms to functionally heal previously irreparable tissues or organs as a result of injury or disease. In regenerative medicine, the use of human adipose tissue-derived stem cells is particularly favourable for cell-based therapies.

In a particular embodiment, said composition or combination can be used in regenerative medicine relating to the regeneration of the musculoskeletal system comprising bones, muscles, cartilage, tendons, ligaments, joints, and other connective tissue that supports and binds tissues and organs together.

In yet another embodiment, said composition or combination can be used in regenerative medicine relating to regeneration of hair follicles, dental pulp, nerves or organs such as liver, kidney, eye, pancreas, intestines, skin.

In yet a further embodiment, said composition or combination can be used in regenerative medicine relating to regeneration of nerve-related injuries and diseases selected from the list comprising: Parkinson's disease, Alzheimer's disease, Huntington's disease, multiple sclerosis, myotrophic lateral sclerosis, multiple system atrophy, (traumatic) spinal cord injury, stroke, hearing loss, (traumatic) brain injury.

As used herein, the term “plastic surgery”, relate to procedures that improve the appearance as well as the function of the human body by restoration, reconstruction, or altering a part of the body. In the context of this application, plastic surgery can be divided into two main categories: reconstructive surgery and cosmetic surgery. Reconstructive plastic surgery is performed to correct functional impairments caused by burns; traumatic injuries, such as facial bone fractures and breaks; congenital abnormalities, such as cleft palates or cleft lips; developmental abnormalities; infection and disease; and cancer or tumours. While reconstructive surgery aims to reconstruct a part of the body or improve its functioning, cosmetic surgery aims at improving the appearance of it such as mammoplasty, eyelid surgery, filler injections in general.

In yet a further embodiment, the present invention provides a composition or combination for use in tissue regeneration, burns.

In another particular embodiment, the present invention provides a composition or combination for use in immunosuppression, immunomodulation, anti-inflammation, pain killing, and/or angiogenesis.

In a further embodiment, the present invention provides a composition or combination for use in osteoarthritis, rheumatology, cardiology, gynecology and/or dermatology.

In another specific embodiment, the present invention provides a formulation for use in liposuction or lipofilling.

As used herein, the term ‘lipofilling’ or also termed ‘fat grafting’ refers to a medical procedure whereby fat is taken via liposuction from a part of the body where it is unwanted or unnecessary, and is grafted (i.e. transferred) to another part.

### EXAMPLES

### Example 1 (In Vitro Study)

### Material and Methods

Subcutaneous adipose tissue was harvested from healthy patients undergoing elective liposuction of the abdomen under general anaesthesia using the dry liposuction technique. Lipoaspirate material was obtained without prior infiltration of a tumescent solution using a 3 millimetre (mm) diameter blunt cannula (Mentor, Santa Barbara, CA, USA) in combination with negative pressure aspiration at −1.5 atmosphere (atm). The lipoaspirate was then centrifuged in 10 millilitre (ml) Luer-Lok syringes (Becton Dickinson, Franklin Lakes, NJ, USA) at 3000 revolutions per minute (rpm) (1200 G; Thermo Fisher Scientific, Waltham, MA, USA) for 3 minutes (min) in order to remove oil from burst fat cells caused by the liposuction. After centrifugation, the oil layer was decantated and the aqueous layer was drained out of the syringe. The resulting bottom layer, predominantly composed of adipose tissue, was used for the in vitro study (FIG. 2). The lipoaspirate material was transported to the laboratory in aseptic conditions without cooling for further sterile processing in a laminar flow hood.

### Cell Cultures & Cell Viability Using the Alamar Blue Assay

The lipoaspirate material was cultured in 24-well culture plates (Nunc) (Thermo Fisher Scientific). Each well was filled with 1 ml of lipoaspirate material and 1 ml of cell culture medium until the lipoaspirate material was covered. Cell culture medium was prepared by adding 1% Penicillin/Streptomycin (P/S) (Gibco, Thermo Fisher Scientific) to each of the investigated solutions. Two solutions were compared: 1) NaCl 0.9% (Baxter International Inc., Deerfield, IL, USA), 2) a CH₃COONₐ comprising solution (27 mmol/L of acetate, 140 mmol/L sodium, 5 mmol/L potassium, 1.5 mmol/L magnesium, 98 mmol/L chloride, 23 mmol/L gluconate), at a 1:1 ratio. This setup was repeated for different exposure times: t₀, t₂₄, t₄₈. The plates were incubated at 37° C. on a gyratory shaker (Laboshake) (Gerhardt GmbH & Co, Germany) for the corresponding periods of time, during which culture media were not changed. No additional antibiotics or growth factors were added. Cell viability was assessed immediately (to, i.e. within 3 hours of harvesting), after 24 hours (t24) and 48 hours (t48). The experiment was performed with three patients in triplicate for each time point. Cell viability and proliferation were quantified using the Alamar Blue assay (Invitrogen, Thermo Fisher Scientific).

Cell viability and proliferation were quantified using the Alamar Blue assay (Invitrogen, Thermo Fisher Scientific). At each time point, 300 microlitre (μl) from each well was retrieved and frozen at −18° C. for leptin analysis electrolyte composition analysis. Next, 170 μl of Alamar Blue reagent was added to each well containing the residual 1.7 ml of lipoaspirate material and culture medium (2 ml-300 μl). The well plate was further incubated at 37° C. for 2 hours. Hereafter, approximately 100 μl was retrieved from each well and transferred to a 96-well plate. The fluorescence intensity was performed on the Wallac 1420 Viktor 3™ plate reader (Perkin Elmer Inc., Waltham, MA, USA) at 525 nm (excitation) and 615 nm (emission). Afterwards, the lipoaspirate/Alamar Blue mixture was discarded.

### Results

### Alamar Blue Assay

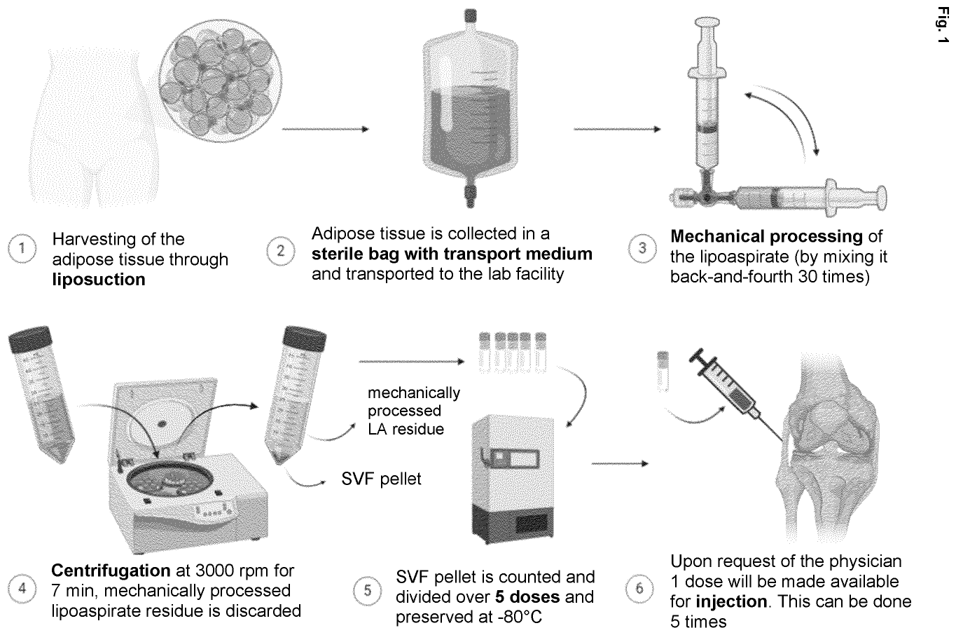

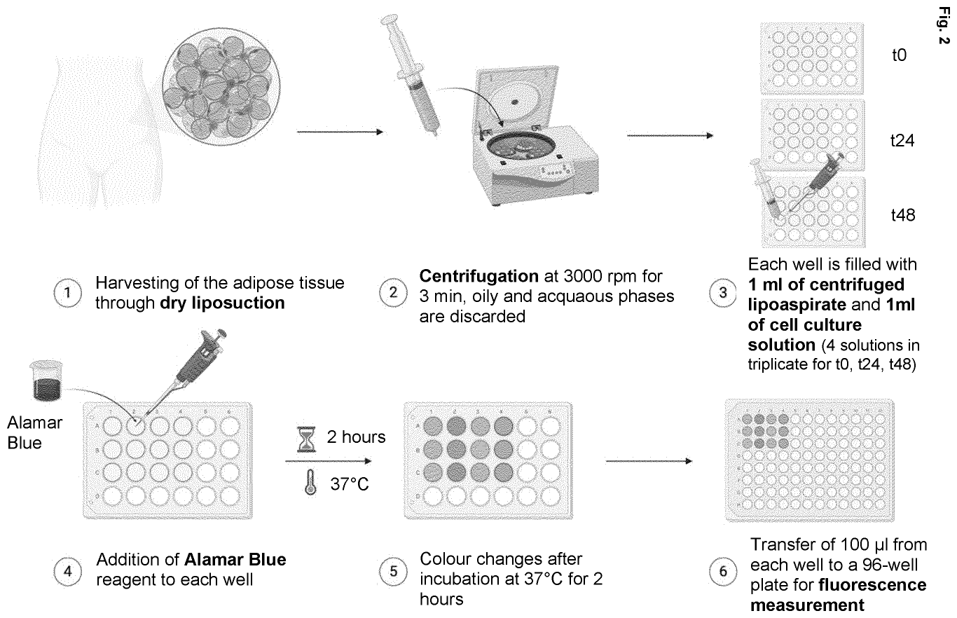

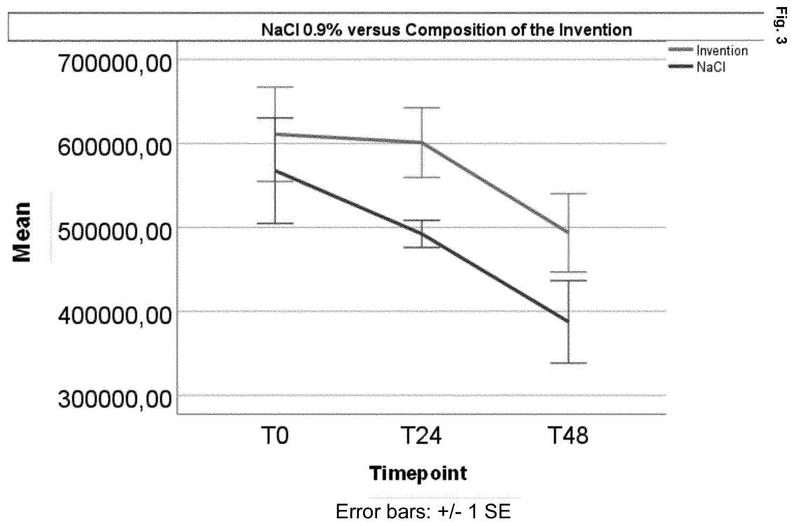

Cell viability and proliferation were quantified using the Alamar Blue assay. The two solutions supported cell viability during two consecutive days (FIG. 3). The initial cell viability immediately after liposuction (to) was high for both groups. After 24 hours (t24) and 48 hours (t48), the CH₃COONₐ comprising solution sustained cell viability at high levels, whereas NaCl 0.9% showed a remarkable decrease of respectively 16% and 25% compared to the group exposed to the CH₃COONₐ comprising solution. The differences between both groups were statistically significant for these time points (p<0.05).

It can be concluded that the CH₃COONₐ comprising solution resulted in the highest cell viability after 24 and 48 hours.

### Example 2 (ASC Culture)

### Material and Methods

Subcutaneous adipose tissue was harvested from healthy patients undergoing elective liposuction of the abdomen under general anaesthesia using (either dry liposuction or liposuction with the proposed wetting solution) (FIG. 1). (1) Lipoaspirate material was obtained without using a 3 millimetre (mm) diameter blunt cannula (Mentor, Santa Barbara, CA, USA) in combination with negative pressure aspiration at −1.5 atmosphere (atm). The lipoaspirate was equally divided into three groups: one group was processed immediately after liposuction (do, i.e. within 3 hours of harvesting) whereas the other two groups were stored for 3 (d3) or 6 (d6) days respectively at 37° C. at a 1:1 ratio. At each time point, three solutions were compared: 1) A CH₃COONₐ comprising solution (27 mmol/L of acetate, 140 mmol/L sodium, 5 mmol/L potassium, 1.5 mmol/L magnesium, 98 mmol/L chloride, 23 mmol/L gluconate) and two conventional wetting solutions i.e., 2) Klein solution and 3) Klein solution enriched with human serum albumin (Alburex 5) (CSL Behring). (2) As such, the lipoaspirate material was collected in a sterile bag together with the corresponding solutions. (3) For mechanical isolation of the SVF from the lipoaspirate, a 20 ml Luer-Lok syringe (Becton Dickinson) filled with 10 ml of lipoaspirate was connected Luer-to-Luer with another 20 ml syringe filled with 10 ml of solution. Next, the lipoaspirate was emulsified by shuffling the mix back-and-forth between the two syringes 30 times. This process was repeated for each solution. (4) The emulsified mixture was then centrifuged at 3000 rpm (1200 G; Thermo Fisher Scientific) for 7 min to obtain the heterogeneous SVF pellet. The overlying fluid and adipose phases were aspirated and discarded. (5) The SVF pellet is counted using e.g. flow cytometry and divided in doses which can be optionally preserved at −80° C. or (6) upon request of the physician, used directly in e.g. a stem cell-based therapy. The remaining stromal cell pellet was resuspended in 1 ml of ASC culture medium and seeded in a T75 culture flask (CellStar) (Greiner Bio-One GmbH, Austria) with an additional 14 ml of ASC culture medium. ASC culture medium was prepared by adding 10% Fetal Bovine Serum (FBS) (Gibco, Thermo Fisher Scientific) and 1% P/S (Gibco, Thermo Fisher Scientific) to Dulbecco's Modified Eagle's Medium (DMEM-GlutaMAX) (Gibco, Thermo Fisher Scientific). The ASCs were incubated at 37° C. and 5% CO2. After 48 hours, culture medium was changed and non-adherent cells were removed. This process was repeated every 3 days. After 4 weeks of culturing, the ASCs were collected. After discarding the culture medium and rinsing the plastic-adhered cell cultures with sterile H₂O, 5 ml of Trypsin-EDTA (Gibco, Thermo Fisher Scientific) was added. Next, the culture flasks were incubated for another 10 min at 37° C. and 5% CO2 to release the adhered cells. Subsequently, the dissociated cells were collected and 5 ml of culture medium (containing 10% FBS) was added to stop enzyme activity. Finally, the dissociated cells were collected for further analysis.

To assess total culture cell count, 20 μl of cell suspension was resuspended with 20 μl trypan blue (Gibco, Thermo Fisher Scientific) in an Eppendorf Safe-Lock microcentrifuge tube (Sigma-Aldrich, St Louis, MO, USA). Cells were counted using a Burker counting chamber (Marienfeld Superior, Thermo Fisher Scientific) and a light microscope (Olympus IX 81 inverted microscope).

### Results

### ASC Culture & Viable Stem Cell Count

Lipoaspirate samples that were mechanically isolated and cultured immediately after liposuction yielded viable ASCs, regardless of the solution used. Lipoaspirate samples that were stored in solution for longer periods of time (i.e. 3 or 6 days) before mechanical isolation did not yield viable ASCs in culture.

Cultured ASCs became spindle shaped after 48 hours and subsequently started rapid expansion (data not shown). After 4 weeks of cell culture, mean cell count was 1.5×10⁶ for CH₃COONₐ comprising solution (do); 2.0×10⁵ for Klein solution (do); 6.0×10⁵ for Klein+ albumin (do) and for 4.5×10⁵ Klein+ albumin (d3). Based on these values, immediate mechanical isolation of lipoaspirate samples with CH₃COONₐ comprising solution resulted in higher stem cell counts after one month of culture.

The inventors also observed beneficial effects of heparin on the mean cell count of CD34+ ASCs and thus clinical manifestation of fat engraftment. The mean collected CD34+ ASCs number was 3,150×10\hat{ }3 cells/ml fat compared to a composition without heparin 1,838×10\hat{ }3 cells/ml fat. This enrichment in CD34+ ASCs show that a composition further comprising heparin increases harvested stem cell numbers significantly prolonging viability and survival of these cells. Fat engraftment was more successful using a composition with heparin compared to a composition without heparin

### CONCLUSION

The findings of this study clearly show that a CH₃COONₐ comprising solution supports the survival of the lipoaspirate in vitro during the first consecutive 48 hours and ultimately result in a higher mean cell count in comparison with NaCl 0.9%. Alternatively, it has been shown that that short-term storage of the lipoaspirate in a CH₃COONₐ comprising solution does not appear to affect the ability of ASCs to grow in culture and clearly yielded the highest numbers of ASCs.

1. A composition comprising CD34+ adipose tissue-derived stem cells (CD34+ ASCs) and sodium acetate (CH₃COONₐ).

2. A composition according to claim 1, further comprising one or more components such as selected from the list comprising sodium, potassium, magnesium, chloride, and/or gluconate; and/or salts thereof.

3. A composition according to any one of claim 1-2, wherein the osmolality ranges from about 270 to about 310 mOsmol/kg, preferably about 291 mOsmol/kg.

4. A composition according to any one of claim 1-3, further comprising a thrombin inhibitor.

5. A composition according to claim 4, wherein the thrombin inhibitor is selected from the list comprising: heparin, desirudin, lepirudin, bivalirudin, hirudin, argatroban, inogatran, melagatran, dabigatran.

6. A composition according to claim 4, wherein the thrombin inhibitor is heparin, in particular unfractionated heparin and/or low-molecular-weight derivates thereof.

7. A composition according to anyone of claim 1-6, further comprising a protein such as a type of globular protein comprising serum albumin, globulins, or a functional equivalents thereof.

8. A composition according to anyone of claim 1-7 for use in human and/or veterinary medicine; in particular for use in stem-cell based therapies, regenerative medicine, and/or plastic surgery.

9. A process for obtaining CD34+ adipose tissue-derived stem cells (CD34+ ASCs), comprising the steps of:
a) Obtaining lipoaspirate material;
b) Adding CH₃COONₐ to said lipoaspirate material of step a;
c) Isolating the stromal vascular fraction (SVF) of said lipoaspirate material of step b;
d) Optionally isolating the CD34+ ASCs from said SVF of step c.

10. A process according to claim 9, wherein step d) comprises:
adding said SVF of step c to a bioreactor of which the surface is pre-treated with a culture medium to promote adhesion of the CD34+ ASCs and subsequently;
detaching said CD34+ ASCs and collecting the cells in a collection medium.

11. A formulation comprising CH₃COONₐ, normal saline, and a vasoconstrictor for use in liposuction or lipofilling.

12. A formulation according to claim 11, further comprising one or more components selected from the list comprising sodium, potassium, magnesium hexahydrate, and/or gluconate; and/or salts thereof.

13. A formulation according to anyone of claims 11-12, further comprising a protein such as a type of globular protein comprising serum albumin, globulins or functional equivalents thereof.

14. A formulation according to anyone of claims 11-13, further comprising a local anesthetic component.

15. A combination, comprising a formulation according to anyone of claims 11-14 and adipose cells, SVF or ASCs.

16. The combination according to claim 15, for use in human and/or veterinary medicine; in particular for use in stem-cell based therapies, regenerative medicine, and/or plastic surgery.
